(19) United States
(12) Patent Application Publication
(10) Pub. No.: US 2026/0221231 A1
(43) Pub. Date: Jul. 30, 2026

(54) ARTIFICIAL INTELLIGENCE (AI)-BASED PROTEIN ENGINEERING SYSTEMS AND METHODS FOR DESIGNING SYNTHETIC PROTEIN SEQUENCES

(71) Applicant: EVOZYNE, INC., Chicago, IL (US)

(72) Inventors: Andrew Ferguson, Chicago, IL (US); Emre Sevgen, Chicago, IL (US); Joshua Moller, Chicago, IL (US); Adrian Lange, Chicago, IL (US)

(21) Appl. No.: 19/143,007

(22) PCT Filed: Jan. 9, 2024
(86) PCT No.: PCT/US24/10805

§ 371 Date: Jun. 25, 2025

Related U.S. Application Data
(60) Provisional application No. 63/479,378, filed on Jan. 11, 2023.

(51) Int. Cl. G16B 40/30; G16B 40/20

(52) U.S. Cl. CPC G16B 40/30; G16B 40/20

(57) ABSTRACT

Artificial intelligence (AI)-based protein engineering systems and methods are disclosed for designing synthetic protein sequences. The AI-based protein engineering systems and methods include defining a region of interest in a low-dimensional latent space, wherein the region of interest comprises a localized feature set determined based on a protein database defining sequences of proteins, and wherein the localized feature set represents a cluster of proteins having one or more target properties corresponding to a selected chemical or biological function of the proteins. The localized feature set of the region of interest is input into a plurality of decoder layers of a multi-layer AI model, and a generating an output from a final decoder layer of the plurality of decoder layers of the multi-layer AI model, wherein the output defines one or more novel synthetic protein sequences designed to invoke the selected chemical or biological function.

### FIELD OF THE DISCLOSURE

The present disclosure generally relates to artificial intelligence (AI)-based protein engineering systems and methods, and, more particularly, to AI-based protein engineering systems and methods for designing synthetic protein sequences.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Proteins are molecular machines that participate in a variety of biological processes, including those that are essential for life. For example, they have the ability to catalyze microsecond biochemical reactions in the body that would otherwise take years. Proteins are involved in transport (the blood protein hemoglobin transports oxygen from the lungs to the tissues), motility (flagella provide sperm motility), information processing (proteins make up signal transduction pathways in cells), and cell regulation cues (like the hormone insulin) fundamental for normal bodily function. Antibodies that provide host immunity are proteins, as are the molecular motors, like kinesin and myosin, responsible for muscle contraction and intercellular transport. When light reaches the eyes, a membrane protein in the eyes called rhodopsin senses the incident photons and, in turn, activates a cascade of downstream proteins to eventually tell the brain what is seen by the eyes. Proteins thus perform highly diverse and specialized functions.

Although proteins exhibit a remarkable array of properties, all proteins are polymers built from only 20 units called amino acids. Every protein is a linear arrangement of amino acids, referred to as the protein sequence. In its native state, the protein molecule itself twists, turns, and folds to generally form an irregular three-dimensional globule. The precise three-dimensional arrangement of the amino acids, called the protein structure, and the interactions between the amino acids give rise to the function of the protein. A model for protein function can be used to derive the functional properties identifying out all the atomic interactions in the protein molecule (i.e., its energetic architecture). Two independent approaches-one based on protein structure and the other based on evolutionary statistics-exist to understand the energetic architecture of proteins.

The structure-guided view has value. For example, the role of binding site residues can be tested by mutagenesis, based on the idea that if critical amino acids are substituted to a residue that disrupts structure or some critical property of the protein (e.g., binding, catalysis, etc.), the protein is predicted to display a decrease in its ability to function. For example, using this approach the importance of amino acids that comprise the interface of protein and ligand has been demonstrated.

However, the principle of spatial proximity described above does not fully capture the determinants of biochemical function. For example, amino acids could interact in complex cooperative ways in the structure to influence binding site function even from a distance, and the structure alone provides no general model for understanding how such cooperativities are arranged. Accordingly, other approaches to protein design are desired. In particular, the optimization space for protein design is too large and complicated to be tractable using only a structure-based approach.

The goal of protein design is to identify novel molecules that have certain desirable properties. This can be viewed as an optimization problem, in which a search is performed for the proteins that maximize given quantitative desiderata. However, optimization in protein space is extremely challenging because the search space is large, discrete, and mostly filled with unstructured, non-functional sequences. Making and testing new proteins are costly and time-consuming, and the number of potential candidates is overwhelmingly large. Accordingly, for the foregoing reasons, there is a need AI-based protein engineering systems and methods for designing synthetic protein sequences.

### SUMMARY

In various aspects, the disclosure provides a multi-layer AI model that enables the design of de novo (novel) proteins without a Multiple Sequence Alignment (MSA). The multi-layer AI model consists of a Transformer augmented by a dimensionality reduction network and a variational autoencoder (VAE) to learn low-dimensional, generative representations of protein families. The multi-layer AI model can be used to generate an output defining one or more novel synthetic protein sequences designed to invoke selected chemical or biological function(s).

In particular, according to one example aspect of the disclosure, an artificial intelligence (AI)-based protein engineering system is disclosed. The AI-based protein engineering system is configured to design synthetic protein sequences. AI-based protein engineering system comprises a memory, a processor communicatively coupled to the memory, and a multi-layer AI model stored in the memory and accessible by the processor. The AI-based protein engineering system further comprise computing instructions configured for execution by the processor and stored in the memory, which when executed by the processor, causes the processor to define a region of interest in a low-dimensional latent space. The region of interest may comprise a localized feature set determined based on a protein database defining sequences of proteins. In addition, the localized feature set may represent a cluster of proteins having one or more target properties corresponding to a selected chemical or biological function of the proteins. The computing instructions of the AI-based protein engineering system may further be configured causes the processor to input the localized feature set of the region of interest into a plurality of decoder layers of the multi-layer AI model. The computing instructions of the AI-based protein engineering system may further be configured causes the processor to generate, from a final decoder layer of the plurality of decoder layers of the multi-layer AI model, an output defining one or more novel synthetic protein sequences designed to invoke the selected chemical or biological function.

According to a further example aspect of the disclosure, an AI-based protein engineering method is disclosed for designing synthetic protein sequences. The AI-based protein engineering method comprises defining a region of interest in a low-dimensional latent space, wherein the region of interest comprises a localized feature set determined based on a protein database defining sequences of proteins, and wherein the localized feature set represents a cluster of proteins having one or more target properties corresponding to a selected chemical or biological function of the proteins. The AI-based protein engineering method further comprises inputting the localized feature set of the region of interest into a plurality of decoder layers of a multi-layer AI model. The AI-based protein engineering method further comprises generating, from a final decoder layer of the plurality of decoder layers of the multi-layer AI model, an output defining one or more novel synthetic protein sequences designed to invoke the selected chemical or biological function.

According to yet a further example aspect of the disclosure, a tangible, non-transitory computer-readable medium storing instructions for designing synthetic protein sequences is disclosed. The instructions, when executed by one or more processors, causes the one or more processors to define a region of interest in a low-dimensional latent space, wherein the region of interest comprises a localized feature set determined based on a protein database defining sequences of proteins, and wherein the localized feature set represents a cluster of proteins having one or more target properties corresponding to a selected chemical or biological function of the proteins. The instructions, when executed by one or more processors, further causes the one or more processors to input the localized feature set of the region of interest into a plurality of decoder layers of a multi-layer AI model. The instructions, when executed by one or more processors, further causes the one or more processors to generate, from a final decoder layer of the plurality of decoder layers of the multi-layer AI model, an output defining one or more novel synthetic protein sequences designed to invoke the selected chemical or biological function.

In accordance with the above, and with the disclosure herein, present disclosure includes improvements in computer functionality or in improvements to other technologies at least because the claims recite, e.g., a multi-layer AI model. That is, the present disclosure describes improvements in the functioning of the computer itself or any other technology or technical field because the multi-layer AI model is able to achieve highly accurate predictions and/or classifications of defining one or more novel synthetic protein sequences without the need for multiple sequence alignments (MSA). This improves over the prior art at least because prior art methods required MSA data, where there is an impact on the underlying computing device memory, processor utilization, and power utilization, associated with storing and processing such MSA data, the construction of an MSA is only possible for evolutionary-related (i.e., homologous) proteins, and its construction can introduce biases and human preconceptions into the data.

The present disclosure further relates to improvements to other technologies or technical fields at least because multi-layer AI model is able, with use of its multi-layer design, to yield properly formed protein sequences from a developed latent space, even from sparse regions which can yield functional, synthetic proteins with over a hundred mutations. Such highly predictive models were previously unachievable in the prior art without the use of data intensive MSAs.

Still further, the present disclosure includes effecting a transformation or reduction of a particular article to a different state or thing, e.g., taking sequences of proteins and transforming or reducing them into real-world proteins by identifying of one or more novel synthetic protein sequences designed to invoke the selected chemical or biological function.

The present disclosure includes specific features other than what is well-understood, routine, conventional activity in the field, and/or otherwise adds unconventional steps that confine the disclosure to a particular useful application, e.g., AI-based protein engineering systems and methods for designing synthetic protein sequences.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1A illustrates an example multi-layer artificial intelligence (AI) model in accordance with various embodiments disclosed herein.

FIG. 1B illustrates a further example multi-layer AI model in accordance with various embodiments disclosed herein.

FIG. 2A illustrates a flowchart or algorithm of an example AI based method for designing synthetic protein sequences in accordance with various embodiments disclosed herein.

FIG. 2B illustrates a flowchart or algorithm of an example AI based method, which continues from the example AI based method of FIG. 2A for designing synthetic protein sequences in accordance with various embodiments disclosed herein.

FIG. 3 illustrates an example AI based system configured to design synthetic protein sequences in accordance with various embodiments disclosed herein.

FIG. 4A illustrates a further example AI based method configured to design synthetic protein sequences in accordance with various embodiments disclosed herein.

FIG. 4B illustrates one or more real-world synthetic proteins based on the output of the one or more novel synthetic protein sequences in accordance with various embodiments disclosed herein.

FIGS. 5A-C illustrate output or otherwise results of an example multi-layer AI model having two inner two VAE layers trained or otherwise configured on a SH3 dataset employing a 6D latent space in accordance with various embodiments disclosed herein.

FIGS. 6A-6C illustrate output or otherwise results of an example multi-layer AI model that allows for alignment-free generative design and engineering of novel synthetic proteins and that demonstrates parity between MSA based and MSA-free methods in accordance with various embodiments disclosed herein.

FIGS. 7A-7C illustrate output or otherwise results of an example multi-layer AI model organized with respect to an example AAAH family by function without needing an alignment in accordance with various embodiments disclosed herein.

FIGS. 8A-8C illustrate output or otherwise results of an example multi-layer AI model regarding example AAAH data output as broken down by phylogeny at the phylum level in accordance with various embodiments disclosed herein.

FIGS. 9A and 9B illustrate output or otherwise results of an example multi-layer AI model with respect to example interpolations in latent space in accordance with various embodiments disclosed herein.

FIGS. 10A and 10B illustrate output or otherwise results of an example multi-layer AI model regarding generative design and designed protein sequences in accordance with various embodiments disclosed herein.

The Figures depict preferred embodiments for purposes of illustration only.

Alternative embodiments of the systems and methods illustrated herein may be employed without departing from the principles of the invention described herein.

### DETAILED DESCRIPTION

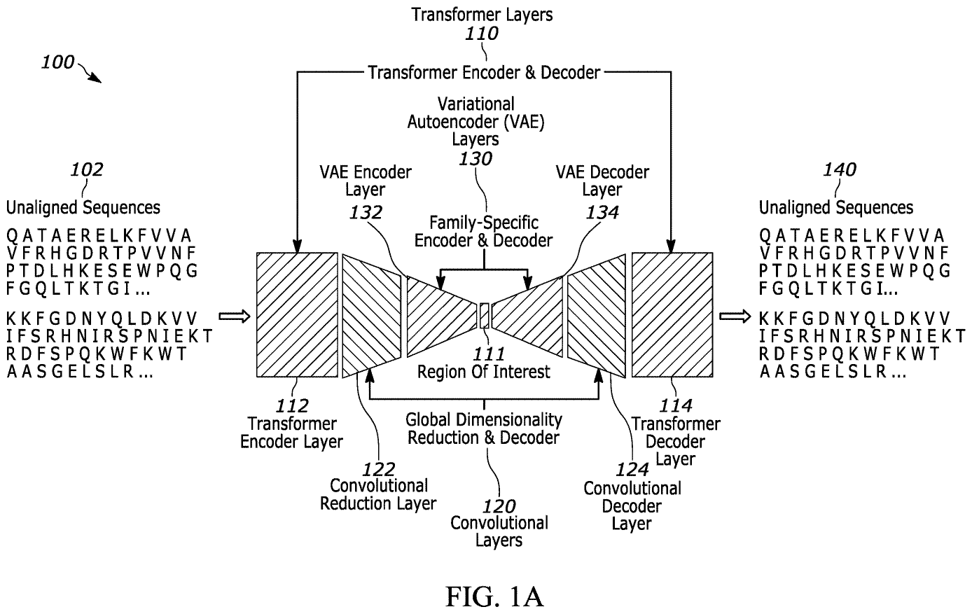

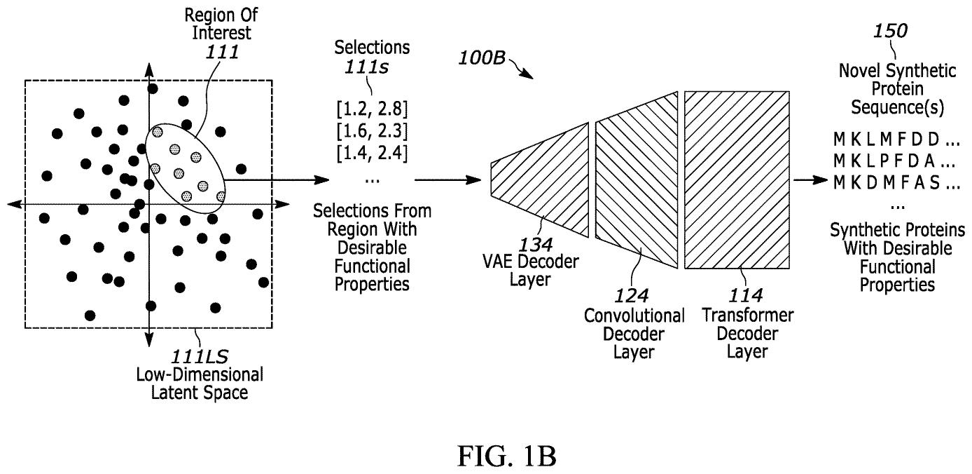

A multi-layer artificial intelligence (AI) model is described herein that enables design of de novo (novel) proteins without a Multiple Sequence Alignment (MSA). The multi-layer AI model comprises a transformer augmented by a dimensionality reduction network and a variational autoencoder (VAE) configured to learn low-dimensional, generative representations of protein families. Diagrams of the model architecture are shown and described herein below for FIGS. 1A and 1B.

The multi-layer AI model described herein overcomes issues with conventional use transformers and VAEs. In particular, transformers are normally very high dimensional and only conditionally generative. Similarly, applications of VAEs to protein engineering have been typically restricted by the requirement for fixed-length training data that require the construction of multiple sequence alignments (MSAs) that are resource intensive, contain biased, and restricted to homologous protein families, and/or that necessitate the use of convolutional or recurrent encoders/decoders that present challenges for underlying computing devices, including increased memory and processor cycles, for learning long-range correlations.

For example, multiple sequence alignments (MSA), which include the process of aligning two or more biological sequences, typically serve as the input to AI models, which are required to provide evolutionary building blocks for protein design. However, MSAs can introduce significant bias, require considerable development time, and can cause overfitting due to bias from the alignment to a single homologous family.

The use of the inventive multi-layer AI model described herein overcomes these challenges and issues. By combining transformer layers and VAE with efficient dimensionality reduction layers in the manner described herein, the multi-layer AI model has the advantages of transformers and VAEs, but without their inherent drawbacks (e.g., impact on underlying computing device memory, processor utilization, and power utilization). The multi-layer AI model leverages transformer layers to eliminate the need for an MSA and/or convolutional or recurrent units. Further, the multi-layer AI model is configured to learn a wide representation of protein sequences trained from a database of proteins having multiple families of proteins. This overcomes previous limitations, which required training an AI model based on a single homologous family of proteins that admit the construction of a MSA or the use of convolutional or recurrent layers that can be challenged in identifying long range correlations. In addition, unlike current transformer-based applications, the description herein based on the multi-layer AI model allows for generation of novel sequences of proteins, while possessing a low-dimensional generative and descriptive latent space, which reduces underlying computing device resources including memory, processor utilization, and power utilization needed to train and use multi-layer AI model.

In this way, the multi-layer AI model described herein provides a generative model for designing or otherwise defining one or more novel synthetic protein sequences without the need for a MSA. The multi-layer AI model is configured to train over large numbers of unrelated protein families to learn generic protein design rules. In addition, the multi-layer AI model is configured to define a fine-tuned or otherwise region of interest for the specific protein family that is the engineering target of interest (e.g., a cluster of proteins having one or more target properties corresponding to a selected chemical or biological function of the proteins). Accordingly, the multi-layer AI model may be used, for example, to design artificial proteins and/or as a generative model to design new proteins for enzyme engineering. Additional and/or different uses of multi-layer AI model are further described herein.

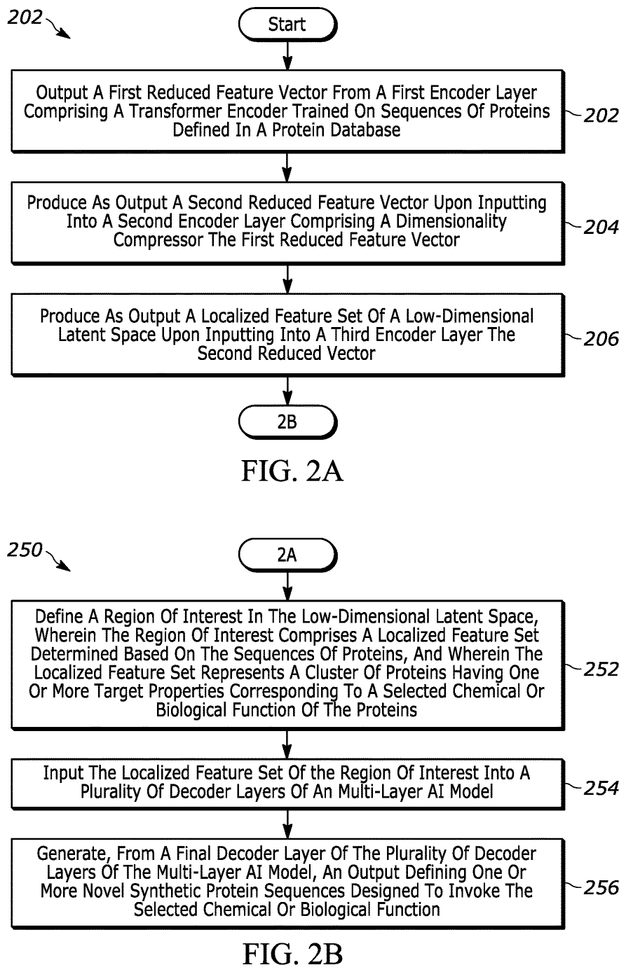

FIG. 1A illustrates an example multi-layer artificial intelligence (AI) model 100 in accordance with various embodiments disclosed herein. In particular, FIG. 1A represents encoding and decoding layers of multi-layer AI model 100, where the encoding layers (e.g., transformer encoder layer 112, convolutional reduction layer 122, and VAE encoder layer 132) are used to train or otherwise configure the multi-layer AI model 100 to executing decoding as performed by the decoding layers (e.g., transformer decoder layer 114, convolutional decoder layer 124, and VAE decoder layer 134). FIG. 2A, further discussed herein below, shows use of a trained version of the model multi-layer artificial intelligence model 100B as may be used in production. The trained version of the model multi-layer artificial intelligence model 100B includes only the decoder layers.

In the example of FIG. 1A, multi-layer AI model 100 is depicted as a large language model (LLM) having symmetrical encoders and decoders, where transformer layers (e.g., transformer layers 110, also referred to herein the transformer encoder and decoder layers) are configured as the outermost layers, and where variational autoencoder layers (e.g., layers variational autoencoder (VAE) layers 130, also referred to herein as family-specific encoder and decoder layers) are configured as the innermost layers. Unaligned protein sequences 102 may be input into transformer encoder layer 112, which may comprise a LLM encoder. The transformer encoder layer 112 outputs values of hidden states representative of the unaligned protein sequences 102. Convolutional reduction layer 122 outputs values of hidden states from transformer encoder layer 112 and compresses these into a reduced or compact representational values using a dimensionality reduction block composed mainly of a series of 1×1 convolutions, but that retains at least a portion of the information provided from the transformer encoder layer 112. These reduced or compact representational values are input into VAE encoder layer 132 into a series of fully connected layers to create a small bottleneck, which may comprise a region of interest 111 comprising proteins of interest. The reduced dimensions (as encoded by the encoding and reduction layers 112, 122, and 132) may then be expanded the same or similar manner in reverse during the decoding process (e.g., by the decoder layers 134, 124, and 114) to reconstruct unaligned protein sequences 140.

In the example of FIG. 1A, unaligned protein sequences 140 show a same value as unaligned protein sequences 102, demonstrating that the encoding and decoding portions of multi-layer AI model 100 operate to encode and reduce a first set of values (unaligned protein sequences 102), and can accurately expand and reproduce the same or similar values (e.g., unaligned protein sequences 140). In various aspects, the dimensionality reduction layers (e.g., layers 112, 122, and 132) of multi-layer AI model 100 may be trained on all proteins, but where the internal block (e.g., region of interest 111) is specific to each protein family. More specifically, as shown for FIG. 1A, multi-layer AI model 100 comprises a plurality of encoder layers comprising a first encoder layer (e.g., transformer encoder layer 112) comprising a transformer encoder trained on sequences of the proteins (e.g., unaligned protein sequences 102) and configured to output a first reduced feature vector. In some aspects, the sequences of proteins upon which the first encoder layer is trained comprises at least one of: a subset of unaligned sequences of proteins or a subset of aligned sequences of proteins. That is, the sequences of proteins may or may not be mutually aligned. Additionally, or alternatively, in various aspects the first reduced feature vector may comprise a fixed-length feature vector.

The first encoder layer may comprise a transformer layer (e.g., transformer encoder layer 112). In various aspects, first encoder layer is configured to generate a high-dimensional feature vector for a given sequence (e.g., unaligned protein sequences 102) that is learned or otherwise obtained for proteins in a given dataset or database. In some aspects, first encoder layer (e.g., transformer encoder layer 112) may comprise a ProtT5 encoder as part of the ProtT5 model, which is a set of computing instructions for performing text-to-text and/or natural language processing (NLP) transformations via transformers for proteins (e.g., a protein language model (pLM)).

Generally, a transformer represents a deep learning architecture employing attention mechanisms to learn many-body and long-range correlated patterns by self-supervised training using masked language modeling. The high-level structure of a transformer employs an encoder block comprising a stack of self-attention layers incorporating position dependent encoding to learn the correlated patterns of amino acid mutations defining the syntax of the protein sequence data within a latent space embedding. The latent space embedding may then be used either as an expressive featurization for high-accuracy downstream functional prediction tasks.

With further reference to FIG. 1A, multi-layer AI model 100 further comprises a second encoder layer (e.g., convolutional reduction layer 122) comprising a dimensionality compressor configured to receive as input the first reduced feature vector (e.g., as output or provided by first encoder layer, e.g., transformer encoder layer 112) and to produce as output a second reduced feature vector. Second encoder layer (e.g., convolutional reduction layer 122) may generate or output second reduced feature vector compressing the first reduced feature vector by applying a one or more 1×1 convolutions to the first reduced feature vector. In this way, second encoder layer comprises a dimensionality reduction layer that efficiently reduces the size of the representation of the first reduced feature vector, as output by the first encoder layer to a smaller version, where the second encoder layer, like the first encoder layer, is trained on the same set of protein sequences (e.g., unaligned protein sequences 102).

With further reference to FIG. 1A, multi-layer AI model 100 further comprises a third encoder layer (e.g., VAE encoder layer 132) configured to receive as input the second reduced vector and to produce as output a localized feature set of a low-dimensional latent space. The latent space may comprise a latent feature space or embedding space comprising a set a set of feature values, where feature values having similar properties are positioned closer to one another in the latent space. The latent space as output by the third encoder layer (e.g., VAE encoder layer 132) is low-dimensional latent space. The latent space is low-dimensional in that it comprises only a few (e.g., three, four, five, or other) dimensions. The feature values may be selected from the dimensional space. Said another way, a third encoder (e.g., VAE encoder layer 132) may comprise protein family-specific VAE that learns a low-dimensional generative representation of that family in the reduced dimensional feature space.

Generally, a VAE is a deep generative models comprising two consecutive neural networks: an encoder compresses a high-dimensional sequence data into a low-dimensional latent space that is then passed to a decoder whose task is to reconstruct the input sequences as the output of the network. The VAE is trained by variational inference to minimize a loss function that typically balances reconstruction accuracy and regularization of the latent space under a prior distribution.

With further reference to FIG. 1A, the low-dimensional generative representation of a given protein family in the reduced dimensional feature space may correspond to a region of interest 111, from which protein sequences may be generated via the decoding layers (e.g., layers 134, 124, and 114) of multi-layer AI model 100. That is, decoder layers (e.g., layers 134, 124, and 114) may be configured to input values from the region of interest 111, as determined by the reduced dimensional feature space, and may further be configured to decompress the values therein to reconstruct a full sequence of proteins (e.g., unaligned protein sequences 140).

As shown for FIG. 1A, multi-layer AI model 100 comprises a series of decoder layers. The decoder layers include a first decoder layer (e.g., VAE decoder layer 134) comprising a generative representation of a protein family. In various aspects, the protein family corresponds to the target properties for a selected chemical or biological function. In addition, the first decoder layer is configured to receive as input a localized feature set (e.g., as determined by the third encoder layer, e.g., VAE encoder layer 132) and to produce as output a first expanded feature vector.

The decoder layers of multi-layer AI model 100 further include a second decoder layer comprising a dimensionally decompressor configured to receive as input the first expanded feature vector and to produce as output, via the dimensionality decompressor (e.g., 5 (e.g., one or more 1×1 convolutions), a second expanded feature vector.

The decoder layers of multi-layer AI model 100 further include a final decoder layer (e.g., transformer decoder layer 114) comprising a transformer decoder configured to receive as input the second expanded feature vector and to produce as output the one or more novel synthetic protein sequences.

The various layers and symmetrical architecture of the multi-layer AI model 100 as shown for FIG. 1A provide various improvements. For example, the convolutional layers 120 between the transformer layers 110 and VAE layers 130 are configured to compress (by convolutional reduction layer 122) the high-dimensional fixed length output of transformer encoder layer 112 before passing such output to the input of the VAE encoder layers 132, and to decompress (by convolutional decoder layer 124) the output of the VAE decoder layer 134 back up to the size of the fixed length transformer featurization prior to passing to the transformer decoder layer 114. During testing of multi-layer AI model 100, incorporation of convolutional layers 120 yielded highly predictive model performance. For example, in some cases, elimination of the convolutional compression/decompression operations as provided by the convolutional layers 120 resulted a less accurate model that could fail to perform adequately for protein engineering tasks. For example, without convolutional layers 120, a given AI model can decode to nonsensical repetitious output sequences (e.g., AAAAAAAAAAAAAAA . . . ) from most of latent space, only managing approximately 5-10% properly decoded sequences in some applications. Furthermore, without convolutional layers 120, only a minority of successful decodes resemble known natural proteins, which severely limits engineering applications. Instead, inclusion of the intermediate convolutional layers 120 for dimensionality reduction yields properly formed sequences for over 95% of decodes from latent space, even from sparse regions which can yield functional, synthetic proteins with over a hundred mutations.

FIG. 1B illustrates a further example multi-layer AI model 100B in accordance with various embodiments disclosed herein. In various aspects, multi-layer AI model 100B represents a production-ready model, configured for deployment or installation on an underlying computing device, and further configured to receive inputs (e.g., one or more target properties corresponding to a selected chemical or biological function of the proteins) to produce predicted and/or classification based outputs (e.g., novel synthetic protein sequences 150).

In various aspects, multi-layer AI model 100B comprises the decoder layers of multi-layer AI model 100 (e.g., layers 134, 124, and 114), where the disclosure for multi-layer AI model 100 applies the same or similarly for multi-layer AI model 100B. For example, in various aspects, after training phase of multi-layer AI model 100 is complete, the encoding layers (e.g., layers 112, 122, and 132) of the model may be discarded and the decoding layers (e.g., layers 114, 124, and 134) may be deployed or otherwise used for generative sequence design. Specifically, as shown for FIG. 1B, regions within the latent space (e.g., region of interest 111), which may contain natural or synthetic sequences with desirable functional properties, may be identified and/or new unexplored regions of sequence space (e.g., an area outside of region of interest 111) predicted to contain sequences with elevated and/or new function may be utilized. In some aspects, such regions are selected, identified, and/or otherwise prospectively resolved using semi-supervised regression models and/or Bayesian optimization. These regions of the latent space are then sampled and decoded to full-length protein sequences through the decoding layers (e.g., layers 114, 124, and 134) of the trained model (e.g., multi-layer AI model 100B) to produce synthetic sequences (e.g., novel synthetic protein sequence(s) 150) predicted to possess selected chemical or biological functions, such as super-natural and/or non-natural functions.

As shown for FIG. 1B, region of interest (e.g., region of interest 111) is defined in the low-dimensional latent space 111LS based on desired functionality being engineered. This can comprise a cluster of proteins within the region of interest 111 having one or more target properties corresponding to a selected chemical or biological function of the proteins. As latent space organizes with respect to function, this region represents a localized luster of proteins with known desirable properties. If no information is present, all of latent space 111LS can be used. In the example of FIG. 1B, low-dimensional latent space 111LS comprises two dimensions reflecting two feature values measured across two dimensions (λ-y dimensions). Once a region of interest (e.g., region of interest 111) is identified, selections 111s from this region may be chosen as samples to be passed as input into first the VAE decoder layer 134. The output of VAE decoder layer 134 may then be passed as input to convolutional decoder layer 124, which output is used as input into transformer decoder layer 114 configured to generate novel, synthetic protein sequences (e.g., novel synthetic proteins sequence(s) 150), for example, as described herein.

FIGS. 1A and 1B illustrate example multi-layer artificial intelligence (AI) models in accordance with the disclosure herein. More generally, the multi-layer AI model as described herein comprises unique layering VAEs and transformers to achieve key characteristics, including at least: (i) accurate learning of the sequence-function relationship, (ii) generative design of sequences under this learned mapping, (iii) fast and transferable model training, and (iv) the capacity unsupervised training over unlabeled sequence data and iterative semi-supervised retraining over labeled data. Generally, the architecture of the multi-layer artificial intelligence (AI) model comprises configuring a VAE layer (e.g., VAE layers 130) between the encoder and decoder stacks (e.g., transformer layers 110) of a transformer protein language model (pLMs). This architecture is enabled by the capacity of the transformer to operate on variable length data but furnish fixed-length latent representations. These representations can be conceived as expressive featurizations of protein sequences that serve as the input to the VAE layers. Within the architecture of the multi-layer AI model(s), the interface between the exterior transformer layers and the interior VAE layers is mediated by simple fully-connected feedforward network layers (e.g., convolutional layers 120) that compress and decompress the transformer layer latent space representation for processing by the VAE layers. The transformer layers (e.g., transformer layers 110) and compression/decompression layers (e.g., convolutional layers 120) may be generic, non-family specific networks that are trained over billions of protein sequence training examples. The VAE of the VAE layers (e.g., VAE layers 130) is a lightweight network that can be trained anew for each particular homologous protein family that is the subject of a design or engineering task. Training of the inner VAE for each specific family is typically fast, and can be the only non-transferable component of the architecture. That is, homologous protein families lie on low-dimensional manifolds of substantially lower dimensionality than that of the transformer latent space, and that the latent space of the transformer layers (e.g., transformer layers 110) can be compressed and decompressed by the VAE with negligible loss of accuracy to furnish a low-dimensional latent space suitable for interpretation, annotation, and guided generative design.

The middle layers (e.g., convolutional layers 120) may be configured to perform partial compression/decompression in a generic, non-family specific manner, and the family-specific VAE layers (e.g., VAE layers 130) can performs the remaining compression/decompression into the family-specific latent space. In this way the multi-layer artificial intelligence (AI) models comprises properties of transformers as generic, transferable, and powerful featurizers capable of learning long-range correlations and operating on variable length sequence data, with the capacity of VAEs to furnish low-dimensional latent embeddings to guide generative sequence design, which can be quickly and iteratively retrained in an unsupervised or semi-supervised fashion.

In some aspects, the multi-layer AI models may be constructed or otherwise generated using the NVIDIA BioNeMo framework for data-driven protein design for computational and experimental applications to protein engineering benchmarks and novel design tasks, including those as described herein. For example, pre-trained transformer encoder and decoder stacks may be used from the approximately 3-billion parameter ProtT5-XL-BFD model trained over the 2.1 billion protein sequences within the (Bulk File Distribution) BFD database and made available within the NVIDIA BioNeMo framework. It should be noted, however, that the multi-layer AI model(s), as described herein, are not dependent on the particular choice of encoder and decoder. For example, training of generic compression and/or decompression blocks may be used to map back and forth between layers of the multi-layer AI model(s). In aspects that use the NVIDIA BioNeMo framework, this can include mapping back and forth between the ProtT5 feature space and a lower-dimensional compression that serves a fixed-length input to a task-specific VAE (e.g., VAE layers 130).

The transformer layers (e.g., transformer encoder layers 110) and compression/decompression layers (e.g., convolutional layers 120) can comprise transferable and generic models that need only be trained once over large libraries of diverse protein sequences and can be conceived of as furnishing expressive fixed-length featurizations of arbitrary proteins from unaligned sequences. In various aspects, only the lightweight VAE (e.g., of VAE layers 13) requires training anew for each protein engineering task and furnishes a smooth, low (e.g., three, four, five, or more) dimensional latent space that provides for conditional generation of synthetic protein sequences with engineered function.

The multi-layer AI model(s) described herein represent a powerful new architecture for data-driven protein engineering that can be deployed to generate synthetic proteins possessing high novelty and functionality for arbitrary design tasks. The only requirement is the availability of sufficient training data—typically ensembles of natural protein sequences—for stable training of a lightweight task-specific VAE to furnish a robust latent space embedding. The multi-layer AI model(s) do not rely on the construction of multiple sequence alignments, which enables the use of robust and expressive attention-based featurizations, and which eliminates the time, labor, and bias associated with alignment construction.

The multi-layer AI model(s) may be used for functional prediction and data-driven design of novel functional sequences after training the model on natural libraries. The multi-layer AI model(s) may be immediately deployed on an underlying computing device within one or more rounds of a machine learning-guided directed evolution (MLDE) campaign by semi-supervised retraining of the VAE on the synthetic sequences and their attendant functional assays. In one embodiment, by virtue of the NVIIDA BioNeMo framework upon which the model is constructed, pre-trained transformer encoder and decoder layers may be used to further increase model training efficiency and retraining of the VAE for typical protein engineering tasks within minutes or hours using commodity NVIDIA GPU cards.

The multi-layer AI model(s) may be used for a panoply of protein engineering and design applications. The transformer and VAE hybrid architecture of the multi-layer AI model(s) is extremely powerful and also generically extensible because it allows incorporation of alternative transformer encoders/decoders and different VAE loss functions and training protocols. In some aspects, the multi-layer AI model may be deployed for the purposes(s) of identification, conditional generation, or design tasks in diverse fields simply by reconfiguring the encoder/decoder blocks for transformer models or layers pre-trained over, for example, nucleic acids, synthetic polymers, small molecules, text, speech, music, or other sequence-based data that reside on low-dimensional manifolds.

### Example Multi-Layer AI Model

In one example, a Multi-layer AI model is based on, or otherwise is configured with or uses, models or data as provided via the NVIDIA BioNeMo framework. In this example, a first portion (e.g., transformer layers 110) of the example multi-layer AI model is a pretrained transformer-based T5 encoder and decoder model referred to as ProtT5nv. This model is available within NVIDIA BioNeMo framework. The ProtT5nv comprises 12 layers, 12 attention heads, a hidden dimension of 768, and a maximum input size of 512. The model employs GeLU activation functions and has a total of ~198M trainable parameters. Training of the model was conducted using masked language modeling employing a 15% masking probability and a dropout of 0.1. Model weights were partially initialized with those taken from a T5 model trained for natural language processing (NLP). Training was conducted over ~46M protein sequences shorter than the maximum 512 input sequence length of the model collated from the UniRef50 database from the May 2022 release. A total of 875K sequences were randomly selected for the validation partition and 4.35K for the test partition. Training was conducted on 224×V100 GPUs for 58 epochs and an inverse square root learning rate schedule employing fused Adam optimization with β1=0.9, β2=0.999, and weight decay=0.01.

A second portion (e.g., convolutional layers 120) of the example multi-layer AI model is a generic dimensionality reduction block that serves to efficiently compress an approximately 300,000-dimensional transformer hidden state into a more parsimonious intermediate-level representation. These intermediate layers are also pretrained on large protein databases and do not require significant changes per protein family. In this example, these layers were pretrained using a mean squared error (MSE) reconstruction objective on Uniprot data. This second portion comprises several stacks of dimensionality reduction layers, where each layer comprises (i) 1×1 convolutions, (ii) LayerNorm, (iii) GeLU activations, and the filter size is incremented at each step. In this example, three layers were used with filter sizes of 512, 256, and 64 in the encoding side and 256, 512, and 768 in the decoding side. These layers serve as a generic dimensionality reduction which are very parameter efficient, fast to train, and do not suffer from significant loss in information content. When trained and validated on large datasets of proteins, reductions of 16 times or more are possible without any noticeable degradation of reconstruction quality. In the current example implementation, the full output of the transformer hidden state, including the positions corresponding to padding tokens, are compressed to create a resultant 32,768-dimensional intermediate representation serves as a much more compact starting point for the family-specific VAE layers (e.g., VAE layers 130) to operate upon and allows for better latent space generation with fewer total network parameters.

A third portion (e.g., VAE layers 130) of the example multi-layer AI model is a three layer fully-connected maximum mean discrepancy variational autoencoder (MMD-VAE) employing ReLU activations that takes the flattened output of the dimensionality reduction block and compresses it into a protein family-specific, low-dimensional latent space. Due to its specificity, this network is initialized and trained from scratch for each target protein family of interest for a particular design task and furnishes the functionally organized, low-dimensional, generative manifold from which synthetic proteins are designed.

The intermediate dimensionality reduction layers were pretrained on Uniprot data with using an Adam optimizer with a learning rate of 0.0001 on an MSE objective function with frozen transformer encoder and decoder. The family-specific VAE layers were trained on unaligned sequences that were members of the homologous target family of interest. We randomly masked 15% of input positions and randomly corrupted 20% of the masked positions to different amino acid residues.

FIG. 2A illustrates a flowchart or algorithm of an example AI based method 202 for designing synthetic protein sequences in accordance with various embodiments disclosed herein. AI-based protein engineering method 202 comprises an encoder algorithm used for training or otherwise generating multi-layer AI model 100 and/or multi-layer AI model 100B.

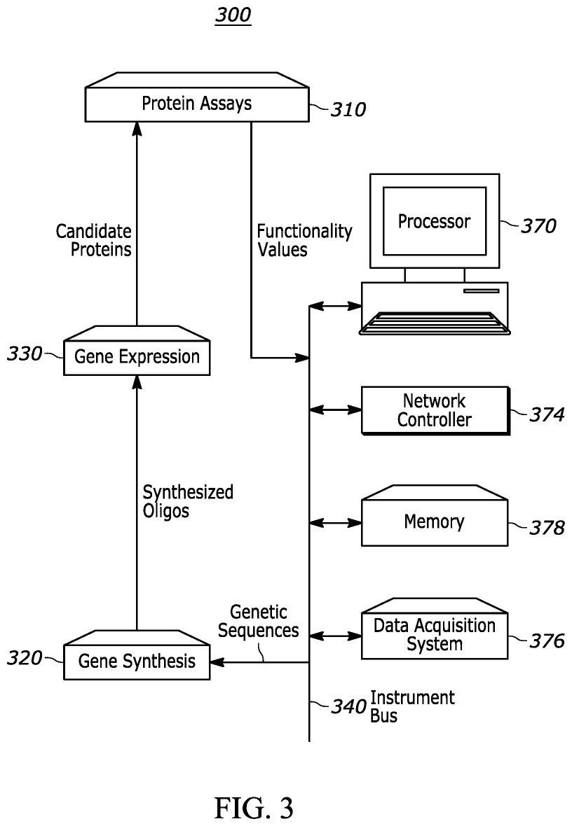

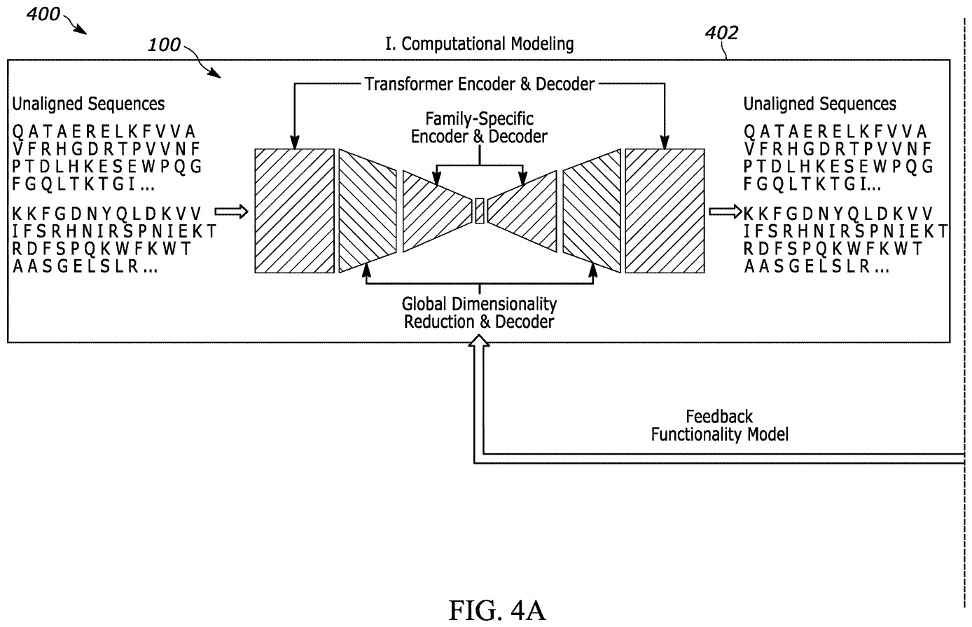

At block 202, AI-based protein engineering method 202 comprises outputting a first reduced feature vector from a first encoder layer (e.g., transformer encoder layer 112) comprising a transformer encoder trained on sequences of proteins (e.g., unaligned sequences 102)) defined in a protein database (e.g., as stored in memory 378 described for FIG. 3).

At block 204, AI-based protein engineering method 202 further comprises producing as output a second reduced feature vector upon inputting into a second encoder layer (e.g., convolutional reduction layer 122) comprising a dimensionality compressor the first reduced feature vector. The second encoder layer may comprise a convolutional filter, which may comprise a 1×1 convolutional filter applied to the first reduced feature vector. It is to be understood, however, that different and/or additional convolutional filters may be applied or executed (e.g., other additional or different combinations, permutations, and/or dimensions of convolutional filters).

At block 206, AI-based protein engineering method 202 further comprises producing as output a localized feature set of a low-dimensional latent space (e.g., low-dimensional latent space 111LS) upon inputting into a third encoder layer (e.g., VAE Encoder Layer 132) the second reduced vector.

FIG. 2B illustrates a flowchart or algorithm of an example AI based method 250, which continues from the example AI based method 200 of FIG. 2A for designing synthetic protein sequences in accordance with various embodiments disclosed herein. AI-based protein engineering method 250 comprises a decoder algorithm used for producing an output (e.g., unaligned protein sequences 140) and/or novel synthetic protein sequences 150) from multi-layer AI model 100 and/or multi-layer AI model 100B.

At block 252 AI based method 250 comprises defining a region of interest (e.g., region of interest 111) in a low-dimensional latent space. In various aspects, a low-dimensional latent space may comprise a reduced dimensional latent space, for example, as illustrated for FIG. 1B, e.g., low-dimensional latent space 111LS. The region of interest (e.g., region of interest 111) may comprise a localized feature set determined based on a protein database defining sequences of proteins. In various aspects, the localized feature set may represent a cluster of proteins (e.g., as shown for region of interest 111) having one or more target properties corresponding to a selected chemical or biological function of the proteins. The localized feature set of the low-dimensional latent space comprises feature values corresponding to the target properties for the selected chemical or biological function. The selected chemical or biological function may correspond to desired chemical or biological functions or properties of the given proteins (e.g., as shown for selections 111s of FIG. 1B). It is to be understood, however, that additional and/or different numbers of latent feature values may be used.

The sequences of proteins in the database may be natural and/or synthetic proteins. That is, at least in some aspects, the sequences of proteins in the protein database may comprise one or more of: natural protein sequences, synthetic protein sequences, protein sequences having known properties, and/or protein sequences having unknown properties. At block 254 AI based method 250 further comprises inputting the localized feature set of the region of interest (e.g., region of interest 111) into a plurality of decoder layers of a multi-layer AI model (e.g., multi-layer AI model 100 and/or multi-layer AI model 100B). The decoder layers may comprise VAE decoder layer 134, convolutional decoder layer 124, and transformer decoder layer 114 as described herein for FIGS. 1A and 1B.

At block 256 AI based method 250 further comprises generating, from a final decoder layer (e.g., transformer decoder layer 114) of the plurality of decoder layers of the multi-layer AI model (e.g., multi-layer AI model 100 and/or multi-layer AI model 100B), an output defining one or more novel synthetic protein sequences (e.g., novel synthetic protein sequences 150) designed to invoke the selected chemical or biological function. In various aspects, novel protein sequences may comprise synthetic protein sequences that may be undefined in the protein database. For example, undefined proteins may include novel proteins and/or proteins having super-natural or non-natural chemical or biological functions. That is one or more novel synthetic protein sequences may comprise protein sequences that are undefined in the protein database (e.g., in memory 378 as described for FIG. 3).

FIG. 3 illustrates an example AI based system 300 configured to design synthetic protein sequences (e.g., novel synthetic protein sequences 150) in accordance with various embodiments disclosed herein. As shown for FIG. 3, AI based system 300 comprise a memory 378 (e.g., a non-transitory computer-readable medium), a processor 370, and a multi-layer AI model (e.g., multi-layer AI model 100 of FIG. 1A and/or multi-layer AI model 100B of FIG. 1B) stored in the memory 378 and accessible by the processor 370. AI based system 300 further comprises computing instructions configured for execution by processor 370 and stored in memory 378, which when executed by the processor, causes the processor to perform the methods and/or algorithms as described herein, including by way of non-limiting example, the method(s) and algorithm(s) for designing synthetic protein sequences as shown and described for FIGS. 2A and 2B.

With further reference to FIG. 3, circuitry and hardware is also shown for a protein optimization system 300 configured to acquire, store, process, and distribute data from the protein assay apparatus 310, the gene synthesis apparatus 320 (also referred to as gene synthesis apparatus/system 320), and the gene expression apparatus 330. The circuitry and hardware include: processor 370, a network controller 374, a memory 378, and a data acquisition system (DAS) 376. The protein optimization system 300 can include a data channel (not shown) that routes results from the respective apparatuses (e.g., the protein assay apparatus 310, the gene synthesis apparatus 320 (also referred to as gene synthesis apparatus/system 320), and the gene expression apparatus 330) to the DAS 376, a processor 370, a memory 378, and a network controller 374. The data acquisition system 376 can control the acquisition, digitization, and routing of the detection data from various sensors and detectors. The processor 370 performs functions including training AI model(s) (e.g., multi-layer AI model 100 of FIG. 1A and/or multi-layer AI model 100B of FIG. 1B), determine regions of interest, controlling the respective apparatuses, and/or preforming other computations or executing instructions as described herein.

Processor 370 can include a central processing unit (CPU) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Processor 370 can execute a computer program including a set of computer-readable instructions that perform various steps of the algorithms(s) and/or methods herein, where the program is stored in memory 378, which may comprise non-transitory electronic memories such as a hard disk drive, CD, DVD, FLASH drive or any other known storage media. Memory 378 can be a hard disk drive, CD-ROM drive, DVD drive, FLASH drive, RAM, ROM or any other electronic storage known in the art. Further, the computer-readable instructions may be provided as a utility application, background daemon, or component of an operating system, or combination thereof, executing in conjunction with a processor, such as a processor from Intel of America or a processor from AMD of America and an operating system, such as Microsoft Windows, UNIX, Solaris, LINUX, Apple, MAC-OS and other operating systems known to those skilled in the art. Further, Processor 370 can be implemented as multiple processors cooperatively working in parallel to perform the instructions.

Network controller 374, such as an Intel Ethernet PRO network interface card from Intel Corporation of America, can interface between the various parts of the protein optimization system 300. Additionally, the network controller 374 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G, 4G, and 5G wireless cellular systems. The wireless network can also be WiFi, BLUETOOTH, or any other wireless form of communication that is known.

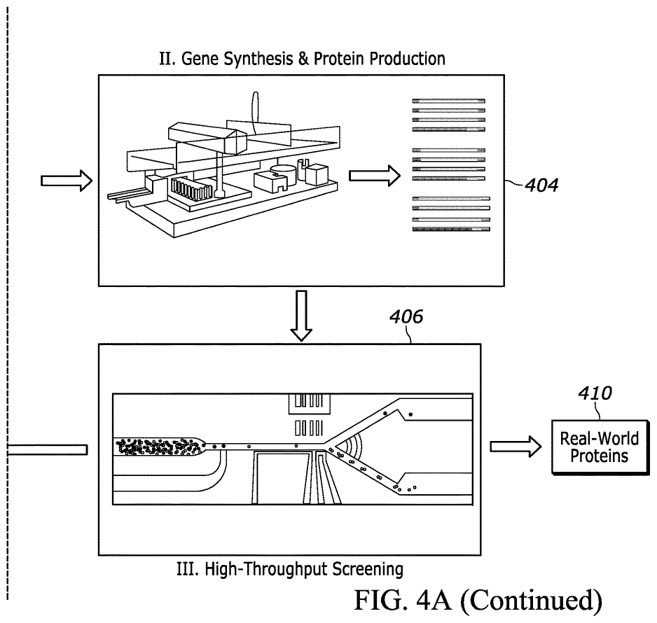

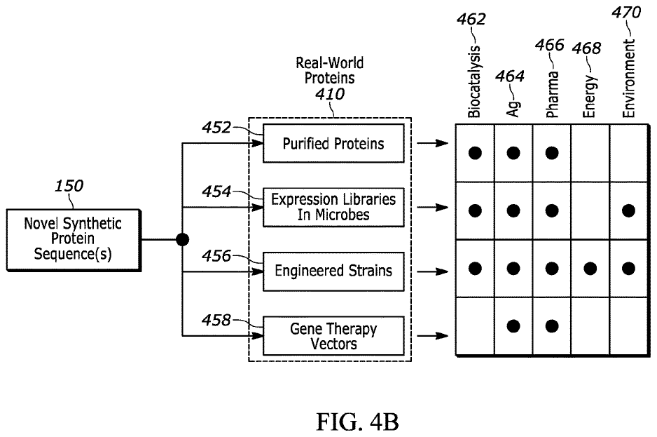

FIG. 4A illustrates a further example AI based method 400 configured to design synthetic protein sequences in accordance with various embodiments disclosed herein. In FIG. 4A, at block 402 a multi-layer AI model (e.g., multi-layer AI model 100 of FIG. 1A and/or multi-layer AI model 100B of FIG. 1B) may be used to perform computational modeling to design synthetic protein sequences (e.g., novel synthetic protein sequences 150) as described herein. At block 404, the synthetic protein sequences may then be provided for gene synthesis and protein production at a laboratory or plant, where real-world proteins are created based on the computationally designed synthetic protein sequences (e.g., novel synthetic protein sequences 150). At block 406, high-throughput functional screening is performed on the real-world proteins to determine quality and effectiveness of their functions. The real-world proteins 410 may then be used as described herein, for example as described for FIG. 4B. The measured values, including their protein sequences, of the high-throughput functional screening may be provided back to the computational modeling (e.g., the sequences are stored in memory 378). In the example implementation of FIG. 4A, the high-throughput functional screening 406 is illustrated using a micro-fluidics apparatus that measures the fluorescence of cells containing respective of the candidate proteins, and directing the cells to different bins based on the measured fluorescence. The measured fluorescence in this implementation is adjusted to be proportional to the protein properties that define the design objectives. Thus, the screen produces data for iterative design of proteins and development of the computational models (e.g., multi-layer AI model 100 of FIG. 1A and/or multi-layer AI model 100B of FIG. 1B).

Protein design based on use of multi-layer AI model 100 and/or multi-layer AI model 100B, may be completely generic and can be applied to the design of synthetic proteins for arbitrary applications where sufficient training data is available to train the given model (e.g., multi-layer AI model 100 and/or multi-layer AI model 100B), where promising regions of the latent space (e.g., low-dimensional latest space 111LS) may be identified for generative design, and where an experimental assay exists to measure resultant protein function. Generally, the training protein sequences (e.g., unaligned sequences 102) can come from natural protein databases or can be synthetic sequences generated by more conventional protocols (e.g., directed evolution, saturation mutagenesis, mutations around promising wild-type candidates). An assay can be specific to each particular functional goal (e.g., thermostability substrate specificity, catalytic activity, neutralization affinity, among others).

FIG. 4B illustrates one or more real-world synthetic proteins 410 based on the output of the one or more novel synthetic protein sequences (e.g., novel synthetic protein sequences 150) in accordance with various embodiments disclosed herein. For example, following output of the one or more novel synthetic protein sequences as provided by multi-layer AI model 100 and/or multi-layer AI model 100B, processor 374 may execute computing instructions to initiate production of one or more real-world synthetic proteins 410 (e.g., as described for FIG. 4A) based on the output of the one or more novel synthetic protein sequences (e.g., novel synthetic protein sequences 150). In some aspects, the one or more real-world synthetic proteins may comprise one or more of: therapeutic proteins, optimization or catalytic enzymes, antibodies, and/or CRISPR-Cas9 proteins.

In the example of FIG. 4B, novel synthetic protein sequences 150 are used to develop real-world proteins 410 including purified proteins 452, expression libraries in microbes 454, engineered strains 456, and/or gene thereby vectors 458. These real-world proteins 410 may then been applied to particular fields or industries, including by way of non-limiting example biocatalysis 462, agriculture 464, pharmaceutical 466, energy 468, and/or environmental 470, as shown for FIG. 4B.

More generally, particular applications of trained models as described herein for synthetic protein design include, but are by no means limited to: (i) the design of novel therapeutic proteins to treat congenital diseases by gene therapy in people possessing defective or malfunctioning versions of particular proteins, (ii) optimization or catalytic enzymes for industrial bioprocesses by enhancing activity, changing substrate specificity, elevating thermostability, etc., (iii) development of “future-proof” antibodies capable of broadly neutralizing activity against possible future strains of infectious pathogens, and/or (iv) development of synthetic CRISPR-Cas9 systems for site-directed genetic modifications.

### Example Applications of the Multi-Layer AI Model

The following applications of the multi-layer AI model (e.g., as described herein) are provided herein to demonstrate latent representations and organizations. The multi-layer AI model is tested in example applications for two protein families: (1) the Src homology 3 (SH3) protein family involved in diverse signaling functions within cells, and the (2) phenylalanine hydroxylase (PAH) enzyme that catalyzes conversion of phenylalanine to tyrosine. The capability of the multi-layer AI model is shown to have meaningful and interpretable latent spaces organizing protein sequences by ancestry and function, to make accurate predictions of protein function from the learned latent space, and to generatively design novel synthetic sequences with function commensurate or superior to natural sequences and with high sequence divergences from the natural training data.

### SH3 Example Multi-Layer AI Model: Overview

The Src homology 3 (SH3) is a family of small beta folds that mediate protein signaling within cells by binding to type II poly-proline peptides with sequences N—R/KXXPXXP-C or N-XPXXPXR/K-C. SH3 domains have evolved to perform a variety of functions within various organisms by evolving differential binding specificities, resulting in a number of distinct paralogs (i.e., homologous proteins performing different functions within the same species) within the SH3 family.

Prior art models require use of an MSA. For example, prior art models have been trained VAEs over an MSA of ~5,300 SH3 homologs to develop a deep generative model for synthetic SH3 design. The prior art VAE learned an unsupervised three-dimensional latent space embedding in which the natural sequences demonstrated an emergent hierarchical clustering by phylogeny and function. The Sho1SH3 domain in Saccharomyces cerevisiae (i.e., baker's yeast) mediates transduction of an osmotic stress signal by binding a Pbs2 ligand that activates a homeostatic response to balance the osmotic pressure by intracellular production of glycerol. A high-throughput in vivo osmosensing assay was developed to measure the relative enrichment of deep sequencing counts of S. cerevisiae Sho1SH3 knockouts into which mutant SH3 genes designed by the VAE were transformed. The normalized relative enrichment (r.e.) is shown to quantitatively report on the binding free energy of the mutant SH3 with the Pbs2 ligand. The assay demonstrated that natural Sho1SH3 orthologs reside within a localized cluster within the VAE latent space and generative design of mutant sequences in the vicinity of this cluster conferred equal or superior high osmolarity protection to wild type Sho1SH3.

By comparison, the multi-layer AI model (e.g., multi-layer AI model 100) is able to learn interpretable latent space embeddings of the SH3 family organized by phylogeny and function without the need for an MSA. The inference of phylogenetic and functional relationships within a learned latent space allows for subsequent data-driven functional protein design.

### SH3 Example Multi-Layer AI Model: Functionality

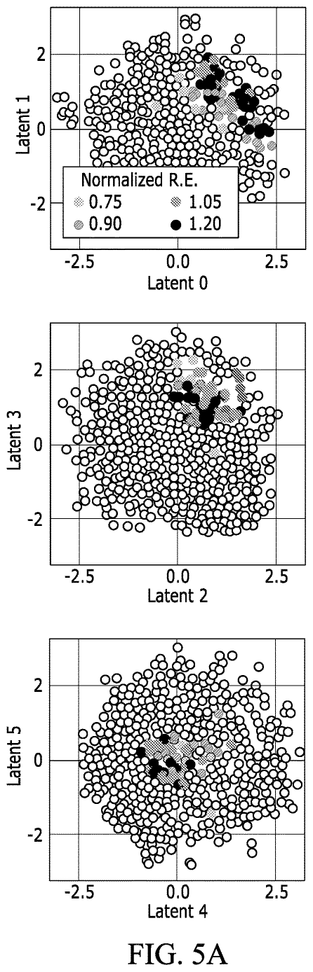

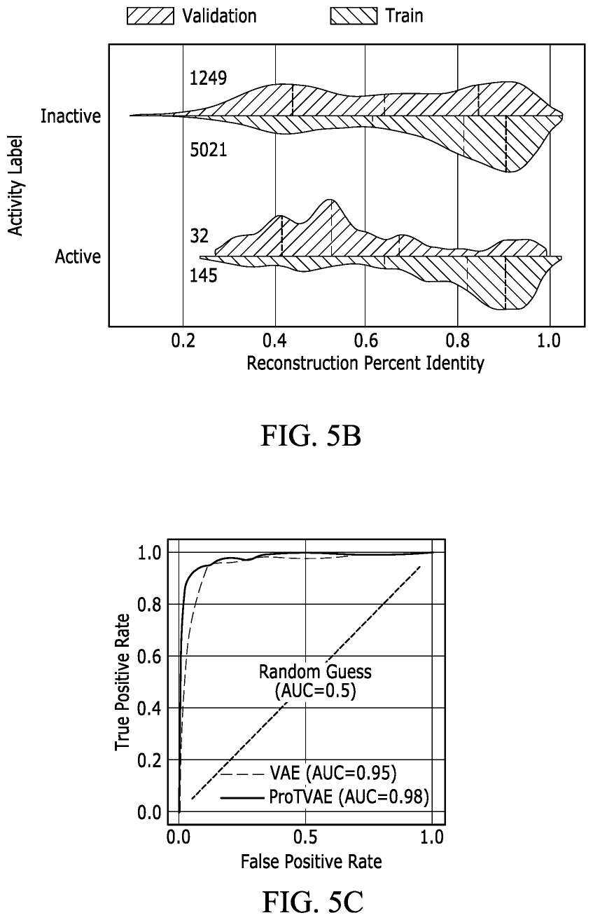

In the present example, the inner two VAE layers (e.g., VAE layers 130) of the multi-layer AI model are trained or otherwise configured on a SH3 dataset employing a 6D latent space. The results are displayed in FIGS. 5A and 5B. As shown for these Figures, in the present example, the multi-layer AI model organizes the SH3 family by functional activity without an MSA. In FIG. 5A, two-dimensional projections of the latent space are shown colored by normalized relative enrichment (r.e.), where darker points correspond to more active sequences. The sequences are clustered in all three projections. In FIG. 5B, reconstruction strength of SH3 sequences are characterized by activity. In FIG. 5B, the distributions for each classification are represented by violin plots separated into those in the training set (bottom-half in blue) and those in the validation set (top-half in green). The number of sequences within each classification and dataset split are printed on the left of the plot. The dashed lines correspond to the 25%, median, and 75% quartile ranges moving from left to right. In FIG. 5C, prediction of functionality via latent space trained classification model is shown. As shown for FIG. 5C, a logistic regression classifier was trained via 5-fold cross validation, using the latent space coordinates and the binary activity labels. The resultant receiver operating characteristic (ROC) of the classifier is shown, demonstrating that the latent space is organized such that protein functionality is localized within the latent space. The green solid line corresponds to the multi-layer AI model, the blue dashed line corresponds to the prior art MSA-based VAE model, and the black dashed line is the null hypothesis. In the legend, the respective area-under-curve (AUC) scores of each of the classifiers is shown.

Further with respect to FIGS. 5A and 5B, the latent space is plotted in 2D projections in three panels, colored by sequences with high r.e. scores. The cutoff based on the dataset is sequences with a normalized r.e.>0.6. Based on the colored projections in FIG. 5A, the high activity sequences are clustered in all dimensions. The activity is featurized into a binary dataset of high and low activity. Using a binary label, the generative potential of the model is evaluated. Due to the low number of high activity sequences in the dataset as can be seen from FIG. 5B, the model has a low average reconstruction of the validation sequences. Using the latent space embedding and the binary labels, a logistic regression was trained via 5-fold cross validation. The results of the regression are shown in the receiver operating characteristic (ROC) curve in FIG. 5C. The multi-layer AI model outperforms the MSA-based VAE model in predicting functional performance with an AUC of 0.98 compared to 0.95. This result validates the capacity of the multi-layer AI model to accurately predict protein function without the requirement for an MSA.

### SH3 Example Multi-Layer AI Model: Phylogeny

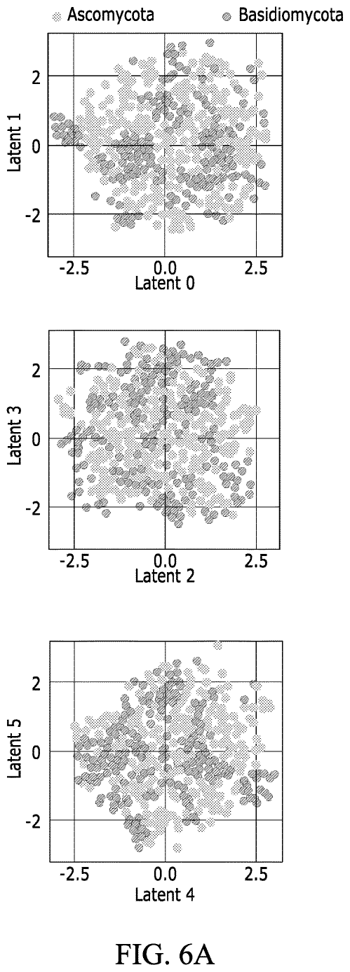

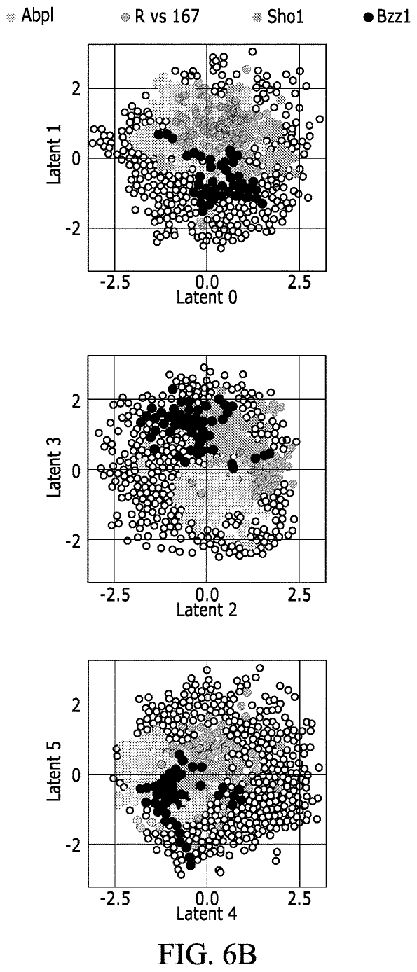

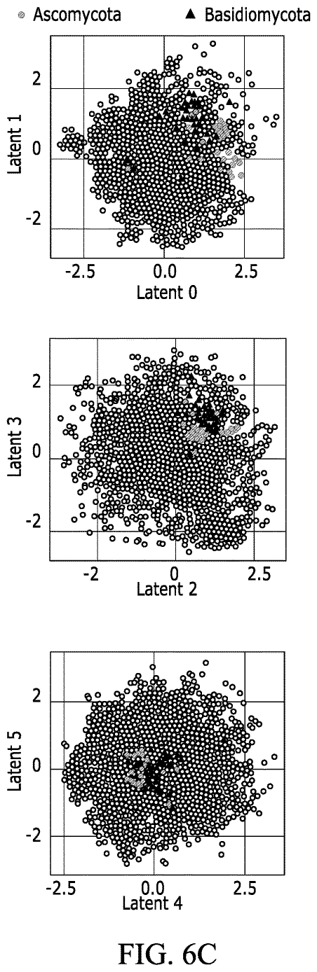

In the present example, the multi-layer AI model separates by paralog group, and then phylogeny is separated within each paralog cluster. The multi-layer AI model allows for alignment-free generative design and engineering of novel synthetic proteins as shown with respect to FIGS. 6A-6C demonstrating the parity between MSA based and MSA-free methods. In FIG. 6A, the multi-layer AI model, which is an MSA-free method, is unable to visually separate phylogeny between Asomycota and Basidiomycota in any latent dimension just as for the MSA based method. Given this result, there is a desire to establish the same hierarchical effect as shown for the MSA-based model. In FIG. 6B, qualitative clustering is demonstrated for the paralog groups of the SH3 family between Abp1, Rvs167, Sho1, and Bzz1. In FIG. 6C, the capability of the model to separate phylogeny within each paralog is exampled. As shown, the Sho1 paralogs are separated by phylogeny, especially in the first two latent dimensions, just as has been shown previously. The multi-layer AI model is able to cluster other annotated paralog groups by phylogeny. The capacity of the multi-layer AI model to learn functional and phylogenetic separation within the latent space without the need for MSAs demonstrates its capacity to learn the correlated patterns of amino acid mutations underpinning the ancestral history and functional performance of sequence ensembles, and allows for alignment-free generative design and engineering of novel synthetic proteins.

Further with respect to FIGS. 6A-6C, the multi-layer AI model hierarchically outputs or provides results organizing SH3 family first by paralog group and then by phylogeny. For example, FIG. 6A shows two-dimensional projections of the latent space colored by two phylogenetic groups, Ascomycota and Basidomycota with no apparent organization. As shown for FIG. 6B, two-dimensional projections of the latent space colored by paralog groups (Abp1 in blue, Rvs167 in orange, Sho1 in green and Bzz1 in yellow) exhibit strong clustering. As shown for FIG. 6C, with respect to the Sho1 paralog group only, the results are colored or otherwise indicated by phylogeny, which demonstrates separation and clustering of Ascomycota and Basidomycota within this paralog group.

### Phenylalanine Hydroxylase (PAH) Example Multi-Layer AI Model: Overview

In a further example, the multi-layer AI model is demonstrated in the context of design of a therapeutic protein. That is, to further evaluate the multi-layer AI model, a second protein family is examined with therapeutic properties. In particular, the family of aromatic amino acid hydroxylases (AAAH), specifically phenylalanine hydroxylase (PAH) is considered. Human PAH (hPAH) is an enzyme that catalyzes the catabolism of one amino acid, phenylalanine, into another, tyrosine, by hydroxylation of the Phe side chain. This reaction is critical in eliminating surplus phenylalanine and producing tyrosine as an essential precursor for the production of hormones, neurotransmitters, and pigments. Starting from a human PAH variant, 2PAH, a psiBLAST (a protein sequence profile search method) program was executed to discover a dataset of homologous proteins, resulting in a dataset of 20,000 sequences. Using annotations from a non-redundant protein database, the dataset was characterized both by substrate specificity and phylogeny. To effectively test the encoding strength of the multi-layer AI model, the inner two VAE layers (e.g., VAE layers 130) of the multi-layer AI model was trained or otherwise configured under an 80-20 training-validation split. All sequences were projected into a 6D latent space for evaluation.

### Phenylalanine Hydroxylase (PAH) Example Multi-Layer AI Model: Substrate Specificity

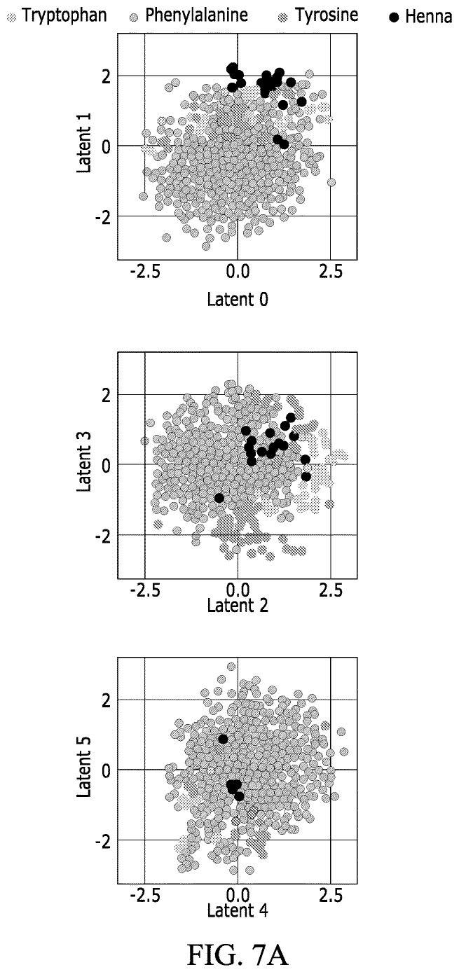

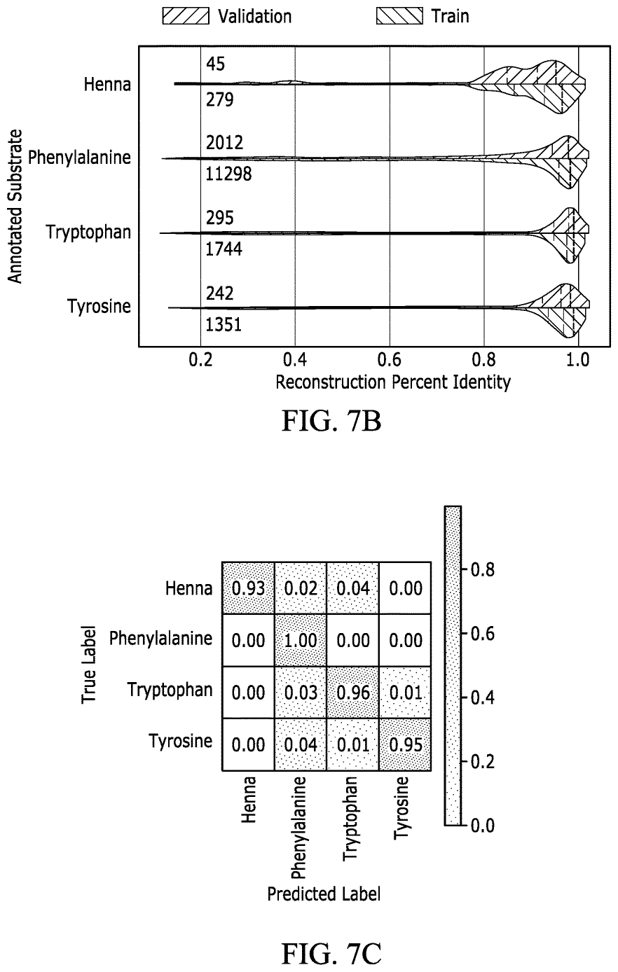

The multi-layer AI model encoding strength is demonstrated with respect to organizing the substrate specificity annotations of the sequences. For example, in FIG. 7A, the latent space is shown in two-dimensional projections, colored by substrate specificity. From FIG. 7A, we observe separation and clustering of the most labeled functional substrates of the AAAH family: tryptophan, phenylalanine, tyrosine, and henna. To demonstrate the generative aspect of the multi-layer AI model, the percent identity of reconstructed sequences is examined. To evaluate this, sequences are passed through the encoder of the multi-layer AI model and then decoded from the latent space, where the percent identity is calculated between the initial sequence and the decoded sequence. The distributions of reconstruction are demonstrated by performing a data split and substrate in FIG. 7B, along with associated counts in each split. In these distributions, there is a strong parity between the training set and validation set in reconstruction strength. For example, henna hydroxylation enzymes consist of the smallest amount of proteins (~1%) yet the validation fraction reconstruction is comparable to the training set median and both are above 90%. Using the latent space encoding, a k-nearest neighbors classifier is trained with k=5 and five-fold cross-validation. In FIG. 7C, the quantitative separation of the latent space embedding via a confusion matrix of the classifier trained on the embeddings is shown. Despite the unbalanced class labels, the multi-layer AI model is able to both generate these sequences with high reconstruction accuracy. In addition, with unsupervised learning, the multi-layer AI model is also able to predict the substrate specificity with high accuracy based on the learned functional organization of the latent space.

Further with respect to FIGS. 7A-7C, the multi-layer AI model produces output or results that organizes the AAAH family by function without needing an alignment (e.g., MSA alignment). As shown for FIG. 7A, two-dimensional projections of the latent space are shown colored by substrate specificity. As shown for FIG. 7B, reconstruction of AAAH family sequences is characterized by substrate specificity. The distributions for each classification are represented by violin plots separated into those in the training set (blue) and validation set (green). The number of sequences within each classification and dataset split are printed on the left of the plot. Dashed lines correspond to the 25%, median, and 75% quartile ranges moving from left to right. As shown for FIG. 7C, prediction of functionality via latent space trained classification model. A k=5-nearest-neighbors classifier was trained via five-fold cross validation, using the latent space coordinates. The resultant confusion matrix of the model is shown for FIG. 7C, demonstrating that the latent space is organized such that protein functionality is localized.

### Phenylalanine Hydroxylase (PAH) Example Multi-Layer AI Model: Phylogeny

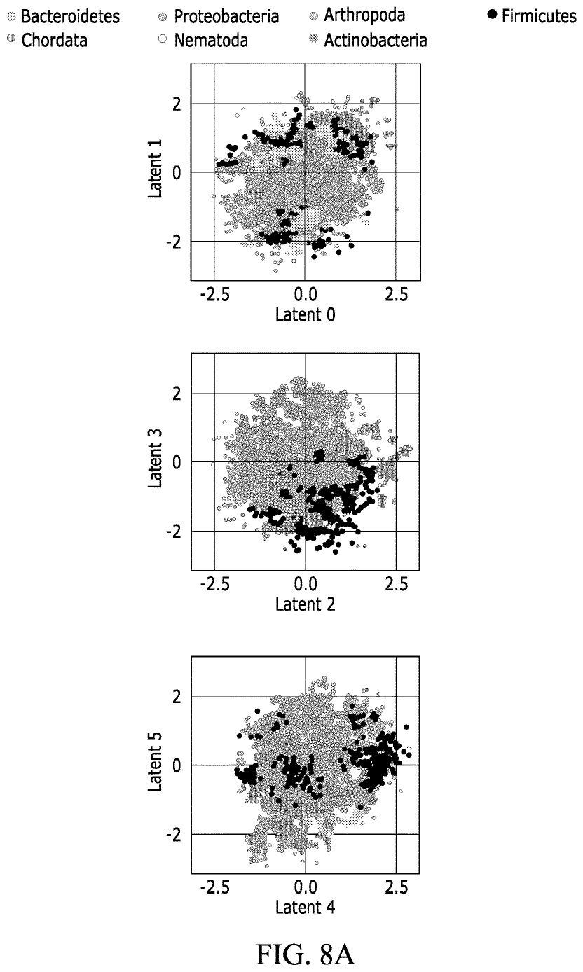

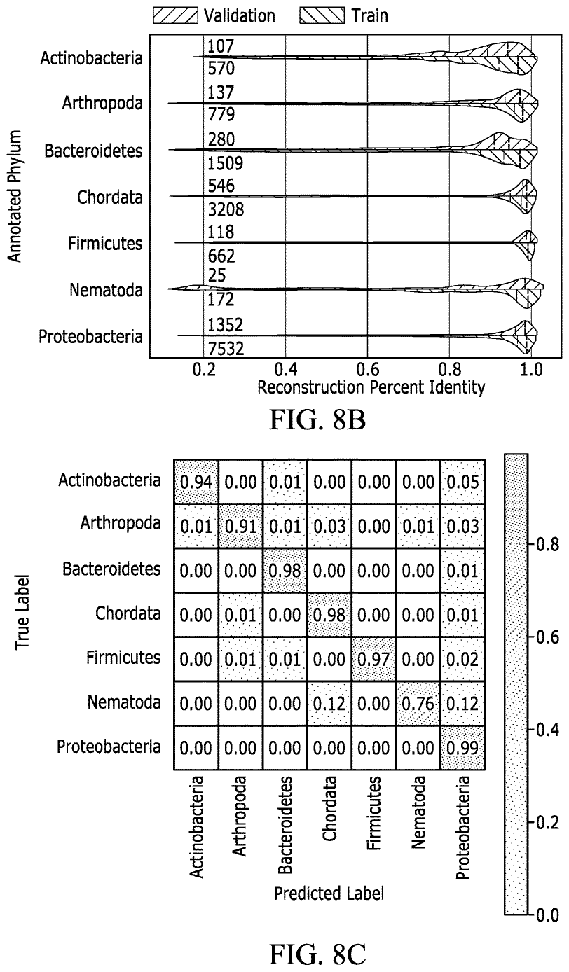

While prediction functionality of the PAH protein is of importance, there are a myriad of phenotypes associated with each protein in the dataset. A key task in the design of therapeutics, for example, is reduction of immunogenic responses. Accordingly, investigation of the predictive capability of the model over multiple tasks may be performed. In the present example, the AAAH dataset is broken down by phylogeny at the phylum level and, using the same latent space, the embedding is annotated by phylogeny. The results are shown for FIGS. 8A-8C. Similar to the substrate specificity results, good clustering and separation are observed for the of the top phylum labels (FIG. 8A). In addition, high reconstruction accuracy is observed of all classes, even though there is a substantial imbalance across the class labels (FIG. 8B). Such a result demonstrates the generative potential of the multi-layer AI model for the de novo design of proteins with specific functionality for a specific host. This is exceptionally useful in, for example, the potential humanization of proteins for therapeutics with specific activity or specificity. Further, by training another k=5-nearest neighbors classifier on the phylum labels with five-fold cross validation, high prediction accuracy can be achieved as shown for FIG. 8C.

Further with respect to FIGS. 8A-8C, the multi-layer AI model's results and/or output may be organized with respect to the AAAH family by phylogeny without needing an alignment. For example, FIG. 8A shows two-dimensional projections of the latent space colored by phylum characterization. Further, FIG. 8B shows reconstruction of AAAH family sequences characterized by phylum. The distributions for each classification are represented by violin plots separated into those in the training set (blue) and validation set (green). The number of sequences within each classification and dataset split are shown in the left of the plot. Dashed lines correspond to the 25%, median, and 75% quartile ranges moving from left to right. In FIG. 8C, prediction of phylogeny via latent space trained classification model is shown. A k=5-nearest-neighbors classifier was trained via five-fold cross validation, using the latent space coordinates. The resultant confusion matrix of the model is shown, demonstrating that the latent space is locally organized such that protein phylogeny is localized in the learned latent space.

### Phenylalanine Hydroxylase (PAH) Example Multi-Layer AI Model: Latent Space Interpolation

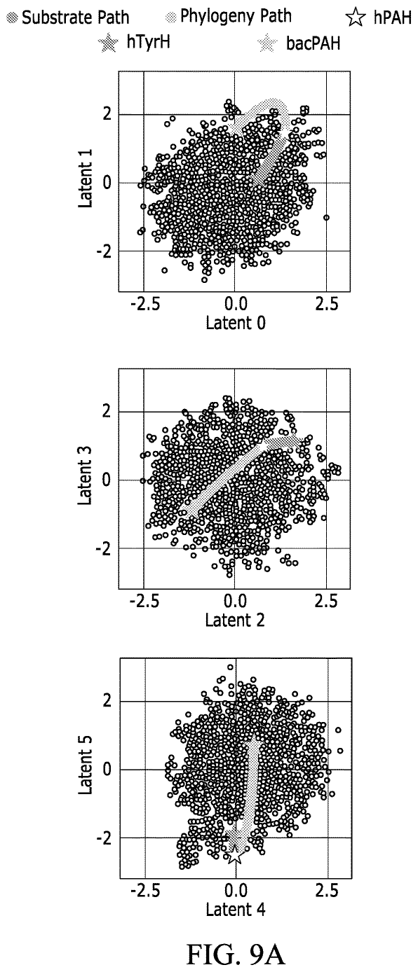

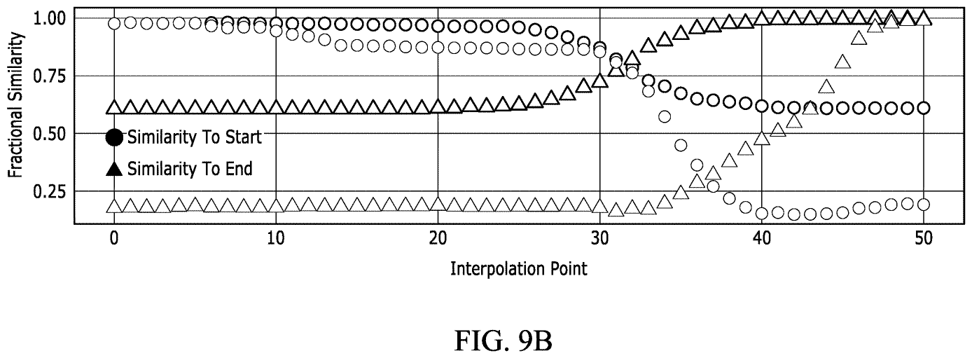

A hallmark of a smooth latent space suitable for optimization and generative design is sensible interpolations. To demonstrate the smoothness of these latent spaces, two separate interpolations are performed with respect to the example multi-layer AI model: one between similar phylogeny but different substrates, and one between different phylogenies acting on the same substrate. In the example, the substrate path was traversed between the 2PAH human PAH (hPAH) and a human tyrosine hydroxylase (hTyrH), while the phylogeny path was between the same hPAH and a flavobacteriacaea PAH sequence (bacPAH). Fifty (50) points were interpolated with spherical linear interpolation (SLERP) and the results of both interpolations are shown in FIG. 9. In FIG. 9A, both interpolations are visualized in the latent space, colored by interpolation path. As shown, there is a longer traversal path for the phylogeny interpolation than for substrates, which is also correlated with larger sequence similarity changes. At each point, the sequence is reconstructed through the decoder of the multi-layer AI model to evaluate the sequence similarity of these novel sequences (FIG. 9B). Despite unique paths of different lengths, there is a smooth transition within both paths that exhibits no sharp transitions. It is also of note that the phylogeny interpolation covers a range of 85% difference in sequence, and yet still results in a smooth interpolation. With this result, the multi-layer AI model demonstrates organization of information with its embedding, but also the ability to generate a smooth representation of unique sequences for generative design and experimental testing.

FIGS. 9A and 9B further demonstrate that traversal in latent space smoothly transitions between sequences with respect to the output or results of the multi-layer AI model. For example, FIG. 9A shows two-dimensional projections of the spherical interpolation in the latent space. There are two paths represented: the phylogeny path, in red, which traverses between a human PAH (hPAH) represented by a white star and a bacterial PAH (bacPAH) represented by a red star, and a substrate specific path, in black, which traverses between the same hPAH and a human tyrosine hydroxylase (hTyrH). FIG. 9B shows a graph showing the steady transition from the first sequence into the second as the interpolation proceeds for both paths. Circles represent the percent identity of the sequences in the path with reference to the start and triangles represent similarity to the end sequence.

### Phenylalanine Hydroxylase (PAH) Example Multi-Layer AI Model: Protein Design

Given the strong predictive performance of the multi-layer AI model as shown herein, evaluate of the model regarding de novo sequence design is further described. Specifically, additional capabilities of the multi-layer AI model are explored to furnish interpretable and generative latent spaces by performing generative design of synthetic human-like PAH proteins for experimental evaluation with a plate-based assay. Specifically, the capabilities of multi-layer AI model are demonstrated to furnish interpretable and generative latent spaces by performing generative design of synthetic human-like PAH proteins for experimental evaluation with a plate-based assay. To generate these sequences, local sampling around hPAH in latent space is performed by fitting a multi-dimensional Gaussian centered around hPAH. Certain sequences are filtered out that were duplicates of natural sequences and that imposed a maximum similarity of any generated sequence to any natural sequence in the training data of 99%. To localize the generated sequences in both latent space and sequence space, a cap is placed on the maximum number of mutations (i.e., substitutions, insertions, deletions) in the generated sequences away from hPAH of 140 of the 333 wildtype positions corresponding to a minimum sequence similarity of 58%. Under these criteria, 190 sequences containing a maximum of 133 mutations away from hPAH and spanning a range of lengths of 223-339 residues were chosen for synthesis and testing.

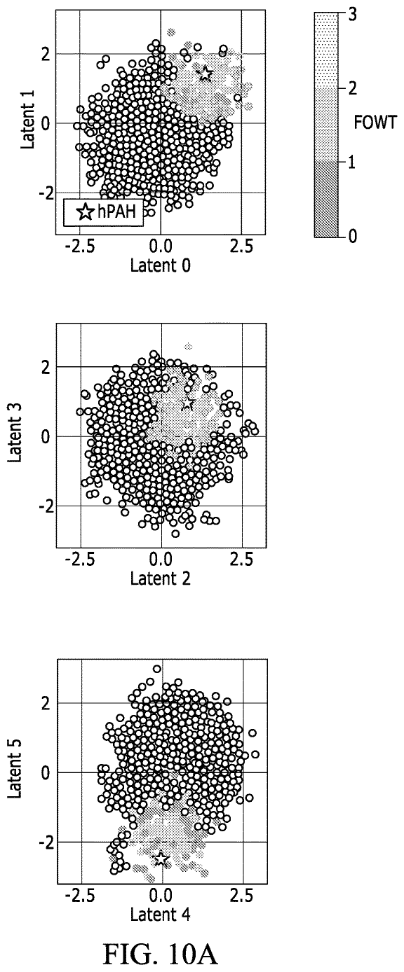

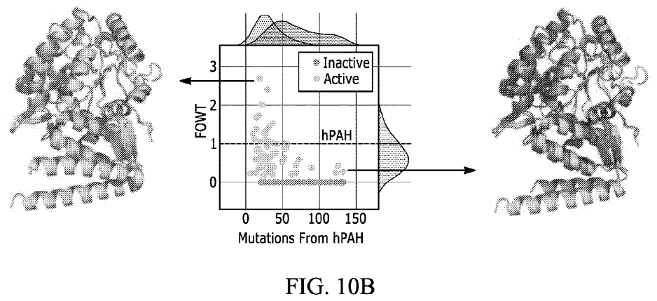

In the example, the assay is designed in 96-well plates using a Biotek plate reader to evaluate the fluorescence of tyrosine produced over a defined time period. The efficiency of catalytic conversion is calculate as the maximum velocity of the reaction, normalized by enzyme concentration in the well. The maximum velocity is calculated by taking the derivative of the curve and finding where it is at a maximum. These values are then normalized by the average hPAH velocity and converted into a fold over wild-type (FOWT) measurement, for easy comparison. The activities of the designed sequences are shown in FIG. 10.

With respect to FIG. 10, generative design of the multi-layer AI model is shown. There were 190 proteins locally designed around human PAH (hPAH) for experimental testing. As shown for FIG. 10A, the latent space is organized by activity in fold-over-wild-type (FOWT). Color corresponds to activity; sequences in black are inactive. hPAH is indicated by a gold star. The training data for the model is shown in gray. FIG. 10B shows activity distribution of the designed sequences as a function of similarity to hPAH. This shows similarity as number of mutations. The predicted structures generated by AlphaFold are shown for the highest-activity synthetic mutant (teal), and the most highly mutated functional synthetic mutant (purple), each aligned with the natural hPAH (green).

In FIG. 10A, the sequences are shown annotated by their relative activity, FOWT, projected into the latent space. The gray points represent the training data to show the overall latent space projections. In black, the inactive sequences are shown, which occupy the same regions of space as the active. All other sequences that are active are shown according to the color bar to the right of the projections. From FIG. 10A, the multi-layer AI model demonstrates is configured to perform MSA-free de novo design of synthetic PAH proteins from a low-dimensional generative and interpretable latent space. Of the proteins assayed, 69 showed activity and 19 were more active than hPAH, with a maximum activity increase of 2.5× that of hPAH. In FIG. 10B, the activities of the generated sequences are shown as a function of the number of mutations from hPAH. Additionally, structures were predicted for the highest active variant and the most mutated variant that showed activity using AlphaFold. The structural models suggest that the synthetic mutants preserve the native fold of the wild-type natural hPAH despite the multi-layer AI model being furnished with no structural information. While the majority of the active sequences are very similar (<30 mutations), the multi-layer AI model is capable of generating highly mutated sequences (>100 mutations, highest active at 130 mutations) that are still functional. This dynamic range of generative design is highly non-trivial from a protein engineering standpoint and shows the engineering power that the multi-layer AI model provides, e.g., not just for its embedding strength, but the ability of the model to generate novel, highly active, and diverse sequences.

### ADDITIONAL CONSIDERATIONS

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. An artificial intelligence (AI)-based protein engineering system configured to design synthetic protein sequences, the AI based system comprising:
a memory;
a processor communicatively coupled to the memory;
a multi-layer AI model stored in the memory and accessible by the processor; and
computing instructions configured for execution by the processor and stored in the memory, which when executed by the processor, causes the processor to:
define a region of interest in a low-dimensional latent space, wherein the region of interest comprises a localized feature set determined based on a protein database defining sequences of proteins, and wherein the localized feature set represents a cluster of proteins having one or more target properties corresponding to a selected chemical or biological function of the proteins,
input the localized feature set of the region of interest into a plurality of decoder layers of the multi-layer AI model, and
generate, from a final decoder layer of the plurality of decoder layers of the multi-layer AI model, an output defining one or more novel synthetic protein sequences designed to invoke the selected chemical or biological function.

2. The AI-based protein engineering system of aspect 1, wherein the plurality of decoder layers of the multi-layer AI model comprises:
a first decoder layer comprising a generative representation of a protein family, wherein the protein family corresponds to the target properties for the selected chemical or biological function, and where the first decoder layer is configured to receive as input the localized feature set and to produce as output a first expanded feature vector;
a second decoder layer comprising a dimensionally decompressor configured to receive as input the first expanded feature vector and to produce as output a second expanded feature vector, and
the final decoder layer comprising a transformer decoder configured to receive as input the second expanded feature vector and to produce as output the one or more novel synthetic protein sequences.

3. The AI-based protein engineering system of aspect 2, wherein the multi-layer AI model comprises a plurality of encoder layers comprising:
a first encoder layer comprising a transformer encoder trained on the sequences of the proteins and configured to output a first reduced feature vector,
a second encoder layer comprising a dimensionality compressor configured to receive as input the first reduced feature vector and to produce as output a second reduced feature vector, and
a third encoder layer configured to receive as input the second reduced vector and to produce as output the localized feature set of the low-dimensional latent space.

4. The AI-based protein engineering system of aspect 3, wherein the first reduced feature vector comprises a fixed-length feature vector.

5. The AI-based protein engineering system of aspect 3, wherein the sequences of proteins upon which the first encoder layer is trained comprises at least one of: a subset of unaligned sequences of proteins or a subset of aligned sequences of proteins.

6. The AI-based protein engineering system of any one of aspects 1-5, wherein the one or more novel synthetic protein sequences comprise protein sequences that are undefined in the protein database.

7. The AI-based protein engineering system of any one of aspects 1-6, wherein the localized feature set of the low-dimensional latent space comprises one to three latent feature values corresponding to the target properties for the selected chemical or biological function.

8. The AI-based protein engineering system any one of aspects 1-7, wherein the computing instructions are further configured, when executed by the processor, to initiate production of one or more real-world synthetic proteins based on the output of the one or more novel synthetic protein sequences.

9. The AI-based protein engineering system of aspect 8, wherein the one or more real-world synthetic proteins comprise one or more of, for example, but not limited to: therapeutic proteins, optimization or catalytic enzymes, antibodies, and/or CRISPR-Cas9 proteins.

10. The AI-based protein engineering system of any one of aspects 1-9, wherein the sequences of proteins in the protein database comprise one or more of: natural protein sequences, synthetic protein sequences, protein sequences having known properties, and/or protein sequences having unknown properties.

11. An artificial intelligence (AI)-based protein engineering method for designing synthetic protein sequences, the AI-based protein engineering method comprising:
defining a region of interest in a low-dimensional latent space, wherein the region of interest comprises a localized feature set determined based on a protein database defining sequences of proteins, and wherein the localized feature set represents a cluster of proteins having one or more target properties corresponding to a selected chemical or biological function of the proteins,
inputting the localized feature set of the region of interest into a plurality of decoder layers of a multi-layer AI model, and
generating, from a final decoder layer of the plurality of decoder layers of the multi-layer AI model, an output defining one or more novel synthetic protein sequences designed to invoke the selected chemical or biological function.

12. The AI-based protein engineering method of aspect 11, wherein the plurality of decoder layers of the multi-layer AI model comprises:
a first decoder layer comprising a generative representation of a protein family, wherein the protein family corresponds to the target properties for the selected chemical or biological function, and where the first decoder layer is configured to receive as input the localized feature set and to produce as output a first expanded feature vector;
a second decoder layer comprising a dimensionally decompressor configured to receive as input the first expanded feature vector and to produce as output a second expanded feature vector, and
the final decoder layer comprising a transformer decoder configured to receive as input the second expanded feature vector and to produce as output the one or more novel synthetic protein sequences.

13. The AI-based protein engineering method of aspect 12, wherein the multi-layer AI model comprises a plurality of encoder layers comprising:
a first encoder layer comprising a transformer encoder trained on the sequences of the proteins and configured to output a first reduced feature vector,
a second encoder layer comprising a dimensionality compressor configured to receive as input the first reduced feature vector and to produce as output a second reduced feature vector, and
a third encoder layer configured to receive as input the second reduced vector and to produce as output the localized feature set of the low-dimensional latent space.

14. The AI-based protein engineering method of aspect 13, wherein the first reduced feature vector comprises a fixed-length feature vector.

15. The AI-based protein engineering method of aspect 13, wherein the sequences of proteins upon which the first encoder layer is trained comprises at least one of: a subset of unaligned sequences of proteins or a subset of aligned sequences of proteins.

16. The AI-based protein engineering method of any one of aspects 11-15, wherein the one or more novel synthetic protein sequences comprise protein sequences that are undefined in the protein database.

17. The AI-based protein engineering method of any one of aspects 11-16, wherein the localized feature set of the low-dimensional latent space comprises one to three latent feature values corresponding to the target properties for the selected chemical or biological function.

18. The AI-based protein engineering method of any one of aspects 11-17 further comprising initiating production of one or more real-world synthetic proteins based on the output of the one or more novel synthetic protein sequences.

19. The AI-based protein engineering method of aspect 18, wherein the one or more real-world synthetic proteins comprise one or more of, for example, but not limited to: therapeutic proteins, optimization or catalytic enzymes, antibodies, and/or CRISPR-Cas9 proteins.

20. The AI-based protein engineering method of any one of aspects 11-19, wherein the sequences of proteins in the protein database comprise one or more of: natural protein sequences, synthetic protein sequences, protein sequences having known properties, and/or protein sequences having unknown properties.

21. A tangible, non-transitory computer-readable medium storing instructions for designing synthetic protein sequences, that when executed by one or more processors, causes the one or more processors to:
define a region of interest in a low-dimensional latent space, wherein the region of interest comprises a localized feature set determined based on a protein database defining sequences of proteins, and wherein the localized feature set represents a cluster of proteins having one or more target properties corresponding to a selected chemical or biological function of the proteins;
input the localized feature set of the region of interest into a plurality of decoder layers of a multi-layer AI model; and
generate, from a final decoder layer of the plurality of decoder layers of the multi-layer AI model, an output defining one or more novel synthetic protein sequences designed to invoke the selected chemical or biological function.

22. An artificial intelligence (AI)-based protein engineering method for designing synthetic protein sequences, the AI-based protein engineering method comprising:
outputting a first reduced feature vector from a first encoder layer comprising a transformer encoder trained on sequences of proteins defined in a protein database,
producing as output a second reduced feature vector upon inputting into a second encoder layer comprising a dimensionality compressor the first reduced feature vector, and
producing as output a localized feature set of a low-dimensional latent space upon inputting into a third encoder layer the second reduced vector.

23. The AI-based protein engineering method of claim 23 further comprising:
defining a region of interest in the low-dimensional latent space, wherein the region of interest comprises the localized feature set determined based on the sequences of proteins, and wherein the localized feature set represents a cluster of proteins having one or more target properties corresponding to a selected chemical or biological function of the proteins,
inputting the localized feature set of the region of interest into a plurality of decoder layers of a multi-layer AI model, and
generating, from a final decoder layer of the plurality of decoder layers of the multi-layer AI model, an output defining one or more novel synthetic protein sequences designed to invoke the selected chemical or biological function.


SEQUENCE LISTING (12 sequences)

SEQ ID NO: 1 - protein, 15 aa, unidentified
AAAAAAAAAAAAAAA

SEQ ID NO: 2 - protein, 48 aa, unidentified
QATAERELKFVVAVFRHGDRTPVVNFPTDLHKESEWPQGFGQLTKTGI

SEQ ID NO: 3 - protein, 49 aa, unidentified
KKFGDNYQLDKVVIFSRHNIRSPNIEKTRDFSPQKWFKWTAASGELSLR

SEQ ID NO: 4 - protein, 48 aa, unidentified; identical to SEQ ID NO: 2

SEQ ID NO: 5 - protein, 49 aa, unidentified; identical to SEQ ID NO: 3

SEQ ID NO: 6 - protein, 7 aa, unidentified
MKLMFDD

SEQ ID NO: 7 - protein, 7 aa, unidentified
MKLPFDA

SEQ ID NO: 8 - protein, 7 aa, unidentified
MKDMFAS

SEQ ID NO: 9 - protein, 48 aa, unidentified; identical to SEQ ID NO: 2

SEQ ID NO: 10 - protein, 49 aa, unidentified; identical to SEQ ID NO: 3

SEQ ID NO: 11 - protein, 48 aa, unidentified; identical to SEQ ID NO: 2

SEQ ID NO: 12 - protein, 49 aa, unidentified; identical to SEQ ID NO: 3